(19) United States
(12) Patent Application Publication
(10) Pub. No.: US 2020/0335192 A1
(43) Pub. Date: Oct. 22, 2020

(54) MEDICATION ADMINISTRATION AND ADHERENCE SYSTEMS AND RELATED METHODS

(71) Applicants: Duke University, Durham, NC (US); North Carolina State University, Raleigh, NC (US)

(72) Inventors: Larry A. Tupler, Durham, NC (US); Yuan-Shin Lee, Raleigh, NC (US); Yi Cai, Raleigh, NC (US)

(21) Appl. No.: 16/661,692

(22) Filed: Oct. 23, 2019

Related U.S. Application Data
(62) Division of application No. 15/766,855, filed on Apr. 9, 2018, now abandoned.
(60) Provisional application No. 62/239,407, filed on Oct. 9, 2015.

(51) Int. Cl. G16H 20/13; A61J 7/00; A61J 7/04

(52) U.S. Cl. CPC G16H 20/13; A61J 7/00; A61J 7/0076; A61J 7/0418; A61J 7/0007

(57) ABSTRACT

Medication administrations and adherence systems and related methods are disclosed. According to an aspect, a medicine administration system includes a case having a drawer that defines compartments for holding medication. The system also includes a mobile computing device. The mobile computing device includes a housing configured to attach to the case. Further, the mobile computing device includes a user interface. The mobile computing device includes one or more processors and memory configured to determine contents of each of the compartments. Further, the processor(s) and memory is configured to manage adherence to a prescription based on the contents The processor(s) and memory are also configured to control the user interface to present information to a user about adhering to the prescription and information about location of prescribed medication in the compartments.

### CROSS REFERENCE TO RELATED APPLICATION

This application claims the benefit of U.S. Provisional Patent Application No. 62/239,407, filed Oct. 9, 2015 and titled INTERNET PILL AUTOMATION CONTROLLER (iPAC) AND METHODS OF USE, the disclosure of which is incorporated herein by reference in its entirety.

### TECHNICAL FIELD

The present subject matter relates to medication administration, adherence systems, feedback between the efficacy of medications and their prescribed indications, and related methods.

### BACKGROUND

Poor medication adherence, which can be defined as a lack of patient compliance to take prescribed medicine following a physician's instruction, is a prevailing problem in healthcare around the world. For example, a patient may fail to consume the correct amount or type of medication in accordance with a prescription schedule. This can occur when a patient forgets to take the prescribed medication, becomes diverted due to other priorities, or possesses a lack information about the prescription.

Currently, there are systems available for assisting patients to adhere to their prescribed medication regimens. Based on their functionality and mobility, current systems can be categorized into the following three types: mobile reminder systems, stationary medication systems, and portable medication systems. A mobile reminder system is usually developed as a software application on mobile devices such as a smartphone, tablet computer, or personal digital assistant (PDA). In a typical setting after the medication schedule of a patient is input or downloaded, a reminder is presented to the patient when it becomes the scheduled time to take a specific dose of medicine. The patient can be informed by the mobile computing device via messages, emails, audio, or visual display. After taking each dose of medicine, a patient may be obligated to report the event via the computing device so that the schedule can be monitored by healthcare personnel, who will react when the patient does not adhere to the prescription. However, the self-report mechanism may result in inaccurate dose reportings due to a user's forgetfulness, carelessness, or perhaps dishonesty.

Stationary medication systems typically include a hardware mechanism to manage physical pills in addition to software applications. Such systems may be developed as a cabinet or pillbox, where different pills are manually pre-sorted into containers according to doses or stored in types. Besides the visual and audio displays as in other systems, various techniques are used to manage pills and monitor medication events, including multimodal sensors, radio-frequency identification (RFID), smart materials, and multimedia. Stationary medication systems are inherently not able to provide a comprehensive solution to non-adherence to a prescription, because a patient is unlikely to stay at the location of the stationary medication system at all times.

Portable medication systems have therefore been developed to serve as a complementary system to stationary medication systems, or work as an independent system. However, a portable medication system tends to be constrained by its size and battery life, because people are unlikely to want to carry a bulky pillbox which needs frequent electrical charging. Moreover, the cost of such a system can greatly influence a user's acceptance. Besides individual drawbacks, a common limitation of these systems is that they tend to work in an open-loop way.

In view of the foregoing, there is a need for improved systems and techniques for managing medication prescriptions and for assisting patients and others with prescription adherence.

### SUMMARY

Disclosed herein are medication administration and adherence systems and related methods. According to an aspect, a medicine administration system includes a computing device having a prescription manager configured to store a prescription for administering medication to a patient. The computing device can include a communications module configured to communicate the prescription via a network. The system also includes a medication dispenser including a receptacle configured to receive medication. The medication dispenser can also include a medication dispensing mechanism configured to position the medication for access by a patient. Further the medication dispenser includes a medication manager configured to receive the prescription via the network. The medication manager is also configured to identify the received medication. Further, the medication manager is configured to control the medication dispensing mechanism to position the medication for access by the patient based on identification of the received medication and the prescription.

According to another aspect, a medication dispenser includes a receptacle configured to receive medication. The medication dispenser also includes a medication dispensing mechanism configured to position the medication for access by a patient. Further, the medication dispenser includes a medication manager configured to receive a prescription for administering the received medication to the patient. The medication manager can also identify the received medication. Further, the medication manager can control the medication dispensing mechanism to position the medication for access by the patient based on identification of the received medication and the prescription.

### BRIEF DESCRIPTION OF THE DRAWINGS

The foregoing aspects and other features of the present subject matter are explained in the following description, taken in connection with the accompanying drawings, wherein:

FIG. 1 is a block diagram of an example medicine administration system in accordance with embodiments of the present disclosure;

FIGS. 2A-2D are different views of an example medication dispenser 100 in accordance with embodiments of the present disclosure;

FIG. 3 is a block diagram of another example medicine administration system in accordance with embodiments of the present disclosure;

FIG. 4 is a partial cut-away diagram of an example medication dispenser in accordance with embodiments of the present disclosure;

FIG. 5 is a flow diagram depicting an example method of use of an example medicine administration system in accordance with embodiments of the present disclosure;

FIG. 6 is a flow diagram depicting an example method of use of an example medicine administration system in accordance with embodiments of the present disclosure;

FIG. 7 is a flow diagram depicting another example method of use of an example medicine administration system in accordance with embodiments of the present disclosure;

FIG. 8 is a flow diagram depicting another example method of use of an example medicine administration system in accordance with embodiments of the present disclosure;

FIG. 9 is a diagram depicting an example case and drawer that cooperates with a dispenser and mobile computing device in accordance with embodiments of the present disclosure;

FIGS. 10A and 10B are different views of the case in accordance with embodiments of the present disclosure;

FIG. 11 is a top view of an interior of the case in accordance with embodiments of the present disclosure;

FIGS. 12A and 12B are a front view and a rear view of a smartphone having a case attached thereto in accordance with embodiments of the present disclosure;

FIGS. 13A and 13B are top views of the case and smartphone;

FIG. 14 is a flow diagram of a patient using a wearable device in accordance with embodiments of the present disclosure; and

FIG. 15 is a diagram of a system having a sensor-embedded water cup for medication detection according to embodiments of the present disclosure.

### DETAILED DESCRIPTION

For the purposes of promoting an understanding of the principles of the present disclosure, reference will now be made to various embodiments, and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

In this disclosure, “comprises,” “comprising, containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless otherwise defined, all technical terms used herein have the same meaning as commonly understood by one of ordinary skill in the art to which this disclosure belongs.

As referred to herein, the term “computing device” should be broadly construed. It can include any type of device capable of administering prescriptions and medication as described herein. A particular computing device as described herein is referred to as a “medication dispenser,” which is a device being configured to dispense medication, such as pills, to a patient. In an example, a computing device or a medication dispenser may be mobile. A computing device or a medication dispenser can be a wireless data access-enabled device that is capable of sending and receiving data in a wireless manner using protocols like the Internet Protocol, or IP, and the wireless application protocol, or WAP. This allows users to access information wirelessly. Wireless data access is supported by many wireless networks, including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are World Wide Web browsers with small file sizes that can accommodate the reduced memory constraints of mobile wireless devices.

As referred to herein, the terms “medication” or “medicine” should be broadly construed. Example forms of medication and medicine include, but are not limited to, pill, powder, or liquid.

As referred to herein, a “user interface” is generally a system by which users interact with computing device or medication dispenser. A user interface can include an input for allowing users to manipulate a computing device, and can include an output for allowing the computing device to present information (e.g., electronic text) and/or data, indicate the effects of the user's manipulation, etc. An example of a user interface on a computing device includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device or medication dispenser for interaction. The display object can be displayed on a display screen of a computing device and can be selected by, and interacted with by, a user using the user interface. In an example, the display of the computing device can be a touch screen, which can display the display icon. The user can depress the area of the display screen at which the display icon is displayed for selecting the display icon. In another example, the user can use any other suitable interface of a computing device, such as a keypad, to select the display icon or display object. For example, the user can use a track ball or arrow keys for moving a cursor to highlight and select the display object. As another example, a user can use a point-and-click device, such as a computer mouse, to select the display object.

Operating environments in which embodiments of the present disclosure may be implemented are also well-known. In a representative embodiment, a mobile electronic device, such as an e-book reader, is connectable (for example, via WAP) to a transmission functionality that varies depending on implementation. Thus, for example, where the operating environment is a wide area wireless network (e.g., a 2.5G network, a 3G network, or a 4G network), the transmission functionality comprises one or more components such as a mobile switching center (MSC) (an enhanced ISDN switch that is responsible for call handling of mobile subscribers), a visitor location register (VLR) (an intelligent database that stores on a temporary basis data required to handle calls set up or received by mobile devices registered with the VLR), a home location register (HLR) (an intelligent database responsible for management of each subscriber's records), one or more base stations (which provide radio coverage with a cell), a base station controller (BSC) (a switch that acts as a local concentrator of traffic and provides local switching to effect handover between base stations), and a packet control unit (PCU) (a device that separates data traffic coming from a mobile device). The HLR also controls certain services associated with incoming calls. Of course, embodiments in accordance with the present disclosure may be implemented in other and next-generation mobile networks and devices as well. The mobile device is the physical equipment used by the end user, typically a subscriber to the wireless network. Typically, a mobile device is a 2.5G-compliant device, 3G-compliant device, or 4G-compliant device that includes a subscriber identity module (SIM), which is a smartcard that carries subscriber-specific information, mobile equipment (e.g., radio and associated signal processing devices), a user interface (or a man-machine interface (MMI)), and one or more interfaces to external devices (e.g., computers, tablets, smartphones, phablets, PDAs, and the like). The electronic device may also include a memory or data store.

The presently disclosed subject matter provides a medicine or medication administration system that includes medication dispenser having a medication dispensing mechanism for dispensing medication, such as pills, to a patient. The system can remove virtually all requirements for memory, executive control, and motor execution from the process of medication prescription adherence. In example use, a user such as a patient, caregiver, pharmacist, or healthcare professional (e.g., a nurse) may pour a bottle of pills for each prescription to a patient into the top of the medication dispenser, and all remaining demands may be subsumed. The medication dispenser may store the pills in a sanitary, tamper-proof container. The medication dispenser may also store the medication regimen in its memory. Subsequently, the medication dispenser may automatically dispense the pills according to the regimen.

In accordance with embodiments, the device may determine whether a patient is taking dispensed pills according to the regimen. In response to determining that the patient has not taken the dispensed pills according to the regimen, the medication dispenser may prompt the patient at intervals to do so. Further, if following prompting the patient does not adhere to his or her regimen, the medication dispenser may notify another, such as a family member or healthcare provider, of noncompliance via a suitable communications technique (e.g., email, text, or phone call).

In accordance with embodiments, a medicine administration system as disclosed herein may subsume many or all medication administration functions from the time of pharmacy fulfillment to pill ingestion. In an example, the system may codify the prescription regimen into an electronically maintained schedule and store pills in a sanitary and tamper-proof container. The system may also dispense the prescribed medication in accordance with the regimen and notify the patient of delivery of one or more pills.

A medicine administration system in accordance with the present disclosure may provide a user interface for assisting a patient or a helper with medication regimen adherence. The user interface may be operably configured with a device as described herein. The user interface may include a display. Further, the user interface may assist with pill identification and otherwise orienting the patient or helper to the prescription pills. The user interface may also provide information about whether pills have been removed from a dispensing holder or well and information about whether a user adheres to the prescription. The user interface may also notify the patient of a scheduled pill consumption if a pill was not taken from the dispenser.

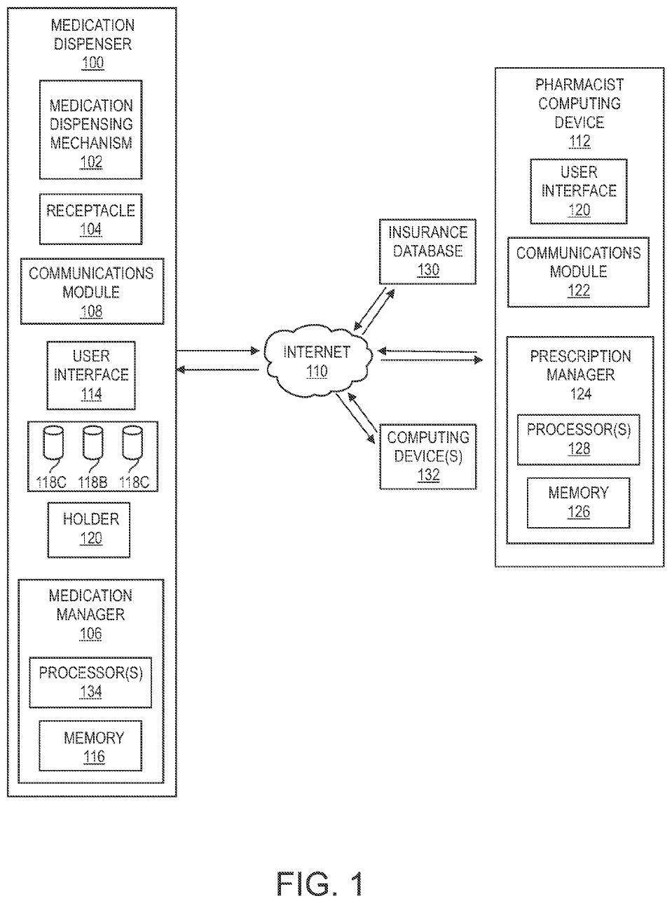

The presently disclosed subject matter is now described in more detail. For example, FIG. 1 illustrates a block diagram of an example medicine administration system in accordance with embodiments of the present disclosure. Referring to FIG. 1, the system includes a medication dispenser 100 that may receive and dispense medicine in accordance with a prescribed regimen. In this example, the medication dispenser 100 is described as being used for dispensing pills, but it should be understood that alternatively the dispenser may be adapted for dispensing medication in other forms such as, but not limited to, powder or liquid. It is noted that in this example the medication dispenser 100 is an electromechanical device having a medication dispensing mechanism 102 configured to position medication for access by a patient. The medication dispenser 100 may also include a receptacle 104 configured to receive medication prior to dispensing the medication. As described in further detail herein, the medication dispenser 100 may also include a medication manager 106 configured to receive a prescription for administering the medication to the patient, to identify received medication, and to control the medication dispensing mechanism 102 to position the medication for access by the patient based on identification of the received medication and the prescription.

In the example of FIG. 1, the medication dispenser 100 is operably connected to the Internet 110 for communication with one or more other components of the medication administration system. Although, it should be understood that the medication dispenser 100 may be operably connected to other components of the medication administration system via any other suitable communications network including a local wireless area network, a cellular network, and/or other networks. The medication dispenser 100 may include a communications module 108 configured for communication with the network. The connection of the medication dispenser 100 allows for remote control of medication dispensing by the medication dispenser 100 as described in further detail herein.

In example operation, a pharmacist or other healthcare personnel may operate a computing device 112 that is suitably configured with a user interface for entry of a medication schedule or prescription. The prescription may identify a schedule for administration of medication to a patient. For example, the prescription may identify one or more of the following: a type of medicine, a dosage amount (e.g., milligrams, numbers of pills), a number of times for the patient to take the medication per day, a time interval between taking doses, and the like. The prescription may also identify the patient. For example, the prescription may identify a name and address of the patient. Prescription and/or other information may be communicated by the pharmacist computing device 112 to the medication dispenser 100 via the Internet 110. The prescription and/or other information may be communicated via any suitable technique for maintaining security. Further, the prescription and/or other information may be received by the communication module 108 and subsequently suitably processed by the medication manager 106 as described by examples provided herein.

Alternative to being entered remotely, prescription information may be entered locally directly into the medication dispenser 100. For example, a user of the medication dispenser 100 may enter the prescription information directly via a user interface 114 of the medication dispenser 100. The user may enter information to tailor the medication delivery timing to accommodate preferred habits, for example, taking a morning dose at a specific, convenient time (e.g., 8:20 a.m.). In another example, prescription information may be entered into the medication dispenser 100 via one or more of a barcode reader, a radio-frequency identification (RFID) tag, or the like. The prescription information may be suitably stored in memory 116 of the medication manager 106.

As will be described in further detail, the medication dispenser 100 may include multiple storage units 118A-118N for use in storing and sorting medication received by the receptacle 104. The medication manager 106 may be configured to use pharmaceutical informatics to derive the size, weight, and/or shape of a pill, for example, which can be used to determine which storage unit 118A-118N is designated for loading. The medication manager 106 can control the medication dispensing mechanism 102 to place medication in its proper storage unit 118A-118N based on identification of the medication. For example, the medication manager 106 may control the medication dispensing mechanism 102 to mechanically receive the medication from the receptacle 104 and to position the medication in storage units 118A-118N based on identification of the medication. As a more particular example, the medication placed in the receptacle 104 may be of different types, and the medication manager 106 may control the medication dispensing mechanism 102 to position medication of the same type in the same storage unit. By directing medication storage into one and only one compartment, the medication dispensing mechanism 102 can accurately deliver a specific medication to a patient. Further, possession of medication in the storage units 118A-118N may be tightly controlled, diminishing the chance of error by the patient or a caregiver. In embodiments, the amount of medication (e.g., number of pills) held by each storage unit 118A-118N may be registered by sensors to further ensure rigorous control over medication inventory. Pills may be dispensed into a holder 120 which allows a patient to grasp the pills. The medication dispenser 100 may have suitable sensors for use in identifying generic and brand names of medication and to retrieve the shape, size, and markings of each pill for information transmission to healthcare personnel, a caregiver, or patient on a display of the medication dispenser 100.

With continuing reference to FIG. 1, computing device 112 may include a user interface 120 for receipt of input from a pharmacist or other operator. The computing device 112 may also include a communications module 122 for communication via the Internet 110. Further, the computing device 112 may include prescription manager 124 configured to store a prescription for administering medication to patient. In an example, the pharmacist may enter the prescription, and the prescription manager 124 may receive and store the prescription in memory 126. The prescription manager 124 may be implemented by any suitable hardware, software, firmware, or combinations thereof. Particularly, the functions of the prescription manager 124 may be implemented by one or more processors 128 executing instructions stored in memory 126.

The medication dispenser 100 may include various modules or components not shown in FIG. 1 for implementing functions described herein. For example, the medication dispenser 100 may include an RFID reader, a barcode reader, motors of the medication dispensing mechanism 102, sensors, lights, speakers, and the like. The medication manager 106 may be configured to suitably operate and receive data from these components.

The system shown in FIG. 1 may include an insurance database 130 and one or more other computing devices 132 in communication via the Internet 110. The insurance database 130 may be used to inform the patient and/or pharmacist of options for generic substitutions for brand name medications. It may also be used to inform insurance companies about level of patient adherence and the success or failure of treatment. The computing devices 132 may be used by healthcare provides, family members, or others for administering the medication dispenser 100 and other functions described herein.

The medication manager 106 may be implemented by any suitable hardware, software, firmware, or combinations thereof. Particularly, the functions of the prescription manager 106 may be implemented by one or more processors 134 executing instructions stored in memory 116.

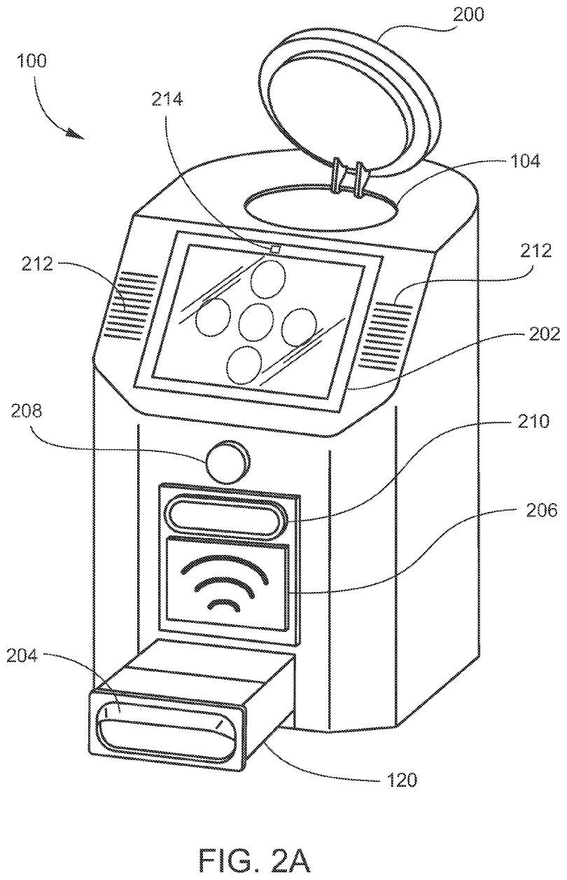

FIGS. 2A-2D illustrate different views of an example medication dispenser 100 in accordance with embodiments of the present disclosure. FIG. 2A illustrates a top perspective view of the medication dispenser 100 in a medication holder open position. FIG. 2B illustrates a top perspective view of the medication dispenser 100 in a medication holder closed position. FIG. 2C illustrates a front view of the medication dispenser 100. FIG. 2D illustrates a top view of the medication dispenser 100.

Referring to FIGS. 2A-2D, the medication dispenser 100 may be used for administering the dispensing of pills to a patient in accordance with a prescription. The medication dispenser 100 may include a receptacle 104 positioned at its top. A rotatable cover 200 may be opened for placement of pills into the receptacle 104. The cover 200 is shown in an open position in FIG. 2A and in a closed position in FIG. 2B. The cover 200 may be closed for covering the receptacle 104. The cover 200 may be locked by combination or key to ensure that the dispenser is tamper-proof. As described herein, pills may be sorted into storage units (such as storage units 118A-118N) after placement in the receptacle 104.

The medication dispenser 100 may include a touchscreen display 202 or GUI, or other suitable user interface, for providing orienting information to the patient or other user. More particularly, the display 202 may indicate the current time, the time of a next scheduled pill delivery or dispensing, the time (e.g., hours and seconds) until the next pill delivery, and prescribed dosage. Further, the display 202 may indicate whether a previous medication has already been taken, thus eliminating the memory burden for the patient.

In accordance with embodiments, the display 202 or another user interface of the medication dispenser 100 can present the prescription details to the patient. For example, the display 202 may display information such as a schedule for administration of the medication, such as a schedule for taking pills and identification of the pills. The pills may be indicated to the patient or another by the pill's name, a text description of the pill, an image of the pill, or another identifier. The display 202 or another user interface may present notification of a timing for taking medication based on the schedule.

The dispenser 100 shown in FIGS. 2A-2D includes a holder 120 that is configured to receive the medication from the medication dispensing mechanism (not shown in FIGS. 2A-2D), and to hold the medication for access by the patient. In this example, the holder 120 is a tray that may be positioned internal to the dispenser 100 for receipt of the medication of the medication dispensing mechanism. After pills are delivered by the dispenser's mechanism to the holder 120, the patient or another may grasp a handle 204 of the holder 120 to pull the holder 120 such that the user may access the pills placed in the holder 120. Alternatively, the holder may be configured to open following the push of a button, or it may be configured to open automatically upon delivery of the medication. It may be configured to close following the push of the same, or a second, button, or it may be configured to close automatically upon removal of all pills delivered by the dispensing mechanism. FIGS. 2A-2D show the holder in position such that the pills can be accessed by the patient.

In accordance with embodiments, the display 202 or another user interface may be configured to receive user input indicating a physical condition of the patient. For example, the patient may use the display 202 to log side effects of use of the medication. Pharmaceutical informatics may be used to provide a checklist displayed to the patient such that the patient may check off side effects known to be associated with the specific medication (e.g., dry mouth). Further, the user interface may provide for a user to enter new side effects not listed in the checklist. The completed checklist, any new entries of side effects, and/or the physical condition of the patient may be stored by the dispenser 100 and suitable to communicate the information to another computing device, such as computing device 112, computing device 132, and/or another computing device via the Internet 110. As a result, individuals and institutions (e.g., pharmaceutical companies), healthcare professions, and other individuals may immediately receive information about side effects of specific prescribed medications, as well as interactions between two or more medications.

In accordance with embodiments, the medication dispenser 100 may include one or more interfaces for communicative coupling to one or more medical monitoring devices. Example medical monitoring devices include, but are not limited to, a blood pressure monitor, a heart rate monitor, a glucose monitor, an activity monitor, and the like. The medication manager, such as the medication manager 106 shown in FIG. 1, may receive measurements of the patient from the medical monitoring device(s) and store the measurement data in memory 116. Further, the medication manager 106 may determine a response of the patient to the medication based on the prescription and the received measurements. The prescription manager 106 may use the communications module 108 to communicate the measurements to another computing device, such as computing device 112 shown in FIG. 1, via a network. Also, abnormalities detected by the equipment may be recorded and communicated to other computing devices. Extreme values denoting an emergent medical event may be transmitted directly via Internet 110 or a wireless communication network to a medical server 306, a physician's computing device 308, and/or an emergency personnel's computing device 310.

In accordance with embodiments, the medication dispenser 100 may include one or more sensors configured to detect whether the patient has taken dispensed medication in accordance with the prescription. In this way, the medication dispenser 100 can collect data for verifying the patient's adherence to a prescription. In an example, a sensor may detect whether a pill has been removed from the holder 120. Further, the medication manager, such as the medication manager 106 shown in FIG. 1, can be configured to use a communications module, such as communications module 108, to communicate the detection to a remote computing device via a network.

The medication manager 106 may receive information from the sensor about when medication is removed and determine whether the medication was removed within a prescribed time period for the patient to take the medication. In response to determining that the medication is not removed within the prescribed time period, then the medication manager 106 may control the user interface 114 to present a prompt for the patient to take the medication in accordance with the prescription. The user interface 114 may be continued to prompt the patient until it is detected that the medication has been taken. Further, in response to determining that the medication is not taken within a prescribed time period and/or after a predetermined number of times that the patient has been prompted, the medication manager 106 may communicate a message to healthcare personnel, a caregiver, or another identified party. In instances whereaupon a critical delay has occurred (e.g., for antibiotics that require a dosage be omitted after a certain delay beyond the indicated dosing time), the dosage can be withheld from delivery until the next scheduled delivery. The user may also manually instruct the iPAC to withhold a medication. Each such instance is logged by the medication manager 106 together with all other information pertaining to medication delivery and adherence (such as removal of medications from the holder).

In accordance with embodiments, a medication dispenser can maintain a record of administering medication. For example, the medication manager 106 of the medication dispenser 100 shown in FIG. 1 can maintain a record of administering of the medication. The medication manager 106 can determine when a patient takes prescribed medication and can report this information to a remote computing device.

In accordance with embodiments, the medication dispenser 100 can include verification equipment configured to verify an identity of the patient for using the medication dispenser. Example verification equipment includes, but is not limited to, an RFID, biometric equipment, facial recognition equipment, voice recognition equipment, and the like. The medication dispenser may include a sensor configured to verify identification of the medication. The sensor may be configured to verify one or more of a weight, a size, a shape, one or more colors, and a marking on the medication.

In accordance with embodiments, the medication manager 106 may determine whether an amount of the medication being stored is below a predetermined level. In response to determining that the amount of the medication being stored is below the predetermined level, the medication manager 106 can control the user interface 114 to present a notification of the predetermined level. The medication manager 106 can control the user interface 114 to present a representation of the medication for the patient. The medication manager 106 can notify a pharmacy or other designated party of the need to refill the medication if refills are permitted according to the prescription. Further, the medication manager 106 can control the user interface 114 to indicate to the patient a schedule for taking the medication. Further, the user interface 114 may be configured to adaptively present the schedule based on a condition of the patient.

In accordance with embodiments, the medication manager 106 can control the user interface 114 to present visual and/or auditory messages to the patient to reinforce adherence to a prescription. For example, an avatar may be displayed that “winks” or “smiles” when medication has been successfully consumed by the patient in accordance with the prescription. Further, verbal reinforcement may include, for example, an avatar thanking a patient for taking his or her pill and reminding him or her when the next pill is due to be taken.

In accordance with embodiments, the medication dispenser 100 may be one of multiple medication dispensers communicatively connected to one another via a network, such as the Internet 110. The medication dispensers can each be associated with a different patient. The prescription manager 124 of the computing device 112 can maintain a database of the patients having a prescription to the same medication. The prescription manager 124 may maintain a database registration of adherence response to specific pills (e.g., a certain pill is not taken in as timely a fashion as another pill because it is too thick, too sticky, has more severe side effects, etc.). Further, the computing device 112 may administer a social network among patients using the medication dispensers. For example, a social network of patients with a particular affliction may be maintained to improve medication administration. Other socially governed influences may also be introduced (e.g., grandchild reminding grandparent or competition with other patients) or data mining. This social network can encourage the patients to be compliant through gaming where competition surrounding adherence can promote timeliness of taking medication. Data mining from the database of networked medication dispensers can be applied to ascertain general principles of adherence across multiple patients and to derive methods for promoting success in reliably consuming medications.

In accordance with embodiments, an electronic medical record for the patient may be maintained at the dispenser or another computing device. For a healthcare personnel such as a nurse, an electronic medication administration record (eMAR) may be maintained.

Referring again to FIGS. 2A-2D, the display 202 can display images of pills scheduled for ingestion to thereby promote user orientation and accuracy of administration. Upon delivery of the medication into the holder 120, a light (e.g., a light emitting diode (LED)) may activate to indicate the medication is ready for retrieval. If, however, the patient does not adhere to the regimen, a re-notification with sound and visual flashing light may be provided to signal that the medication is available for consumption. This process may be repeated at programmed intervals until either the medication has been taken or an alternative adherence-promoting option is executed. As one option, a communication may be sent to designated family members, friends, and/or healthcare personnel via email, wireless transmission (e.g., text message), and/or the like to alert the designated person that a lapse in adherence has occurred. The display or user interface may be customizable in relation to user preferences, including the properties of the avatar.

In accordance with embodiments, the dispenser 100 may store various sensor data in its memory 116. The sensor data may provide information on characteristics of mediation adherence itself. Examples include, but are not limited to, date and time of each pill acquisition from the holder 120, number of reminders provided to the patient, identification of pills characterized by poor adherence (e.g., because of large size or side effects that may not be consciously perceived but nevertheless influence behavior), and the like. An electronic and automated sensor interface of the dispenser 100 may allow for data merging with other medical devices, such a blood pressure monitors or to cognitive assessments to directly link objective signs to prescription indications and medication efficacy. The interface can alert the user to environmental conditions that may compromise the integrity of the medication (e.g., sensors determining that ambient temperature that has exceeded 78 degrees). The computing device 112 through communications module 108 can electronically communicate with prescribing doctors, pharmacies, insurance companies, Express Scripts, and other parties that may be interested in prescription practices and adherence. Also, the dispenser 100 may be seamlessly merged with technology that provides direct, physiological measures of adherence.

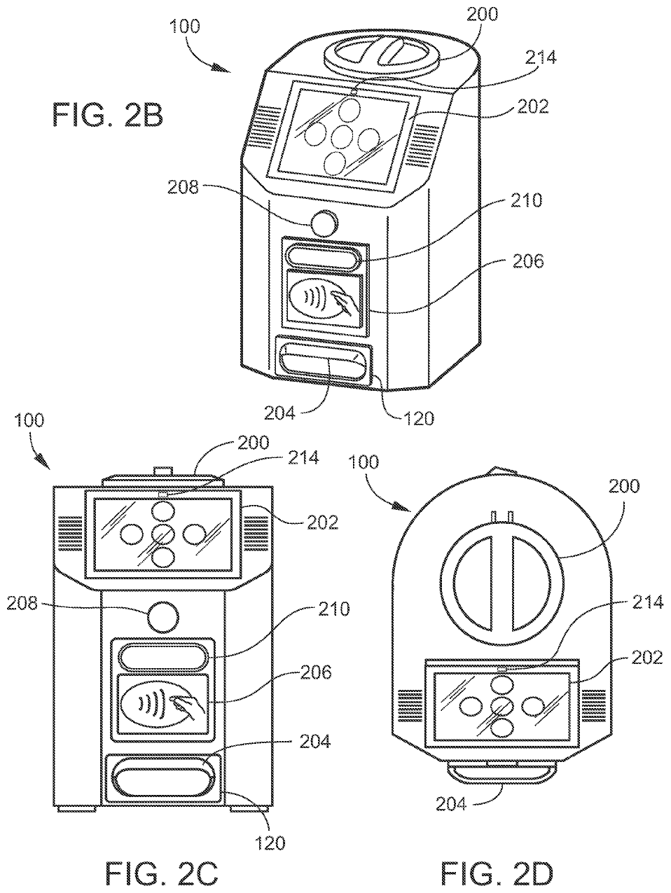

FIG. 3 illustrates a block diagram of another example medicine administration system in accordance with embodiments of the present disclosure. Referring to FIG. 3, the medication dispenser 100 is configured to communicate with other computing devices via the Internet 110. In this example, a patient 300 may carry a smartphone 302 or other mobile computing device. The smartphone 300 may be configured for wireless communication (e.g., BLUETOOTH®) with multiple sensors 304 dispersed on the patient 302. The sensors 304 may be configured to physiological measurements of the patient 302 and to communicate the measurement data to the smartphone 300. The smartphone 300 may in turn communicate the measurement data to the dispenser 100. In an example use, the sensors 304 may detect signals emitted upon contact of coated pills with gastric secretions and textile applications that detect physiological indications of swallowing. As a result, the smartphone 300 and sensors 304 provide a wireless body area network (WBAN). Based on the registration of the identity of a specific pill by the dispenser 100, the informatics of the compound can be used to inform WBAN algorithms. With the enabling of the dispenser 100 at the patient's site, data can be transmitted via Internet 110 or a wireless communication network to a medical server 306, a physician's computing device 308, and/or an emergency personnel's computing device 310. Lapses in adherence at the level of pill ingestion can be registered and communicated. Different sources of information input can be combined to increase the level of confidence in adherence detection.

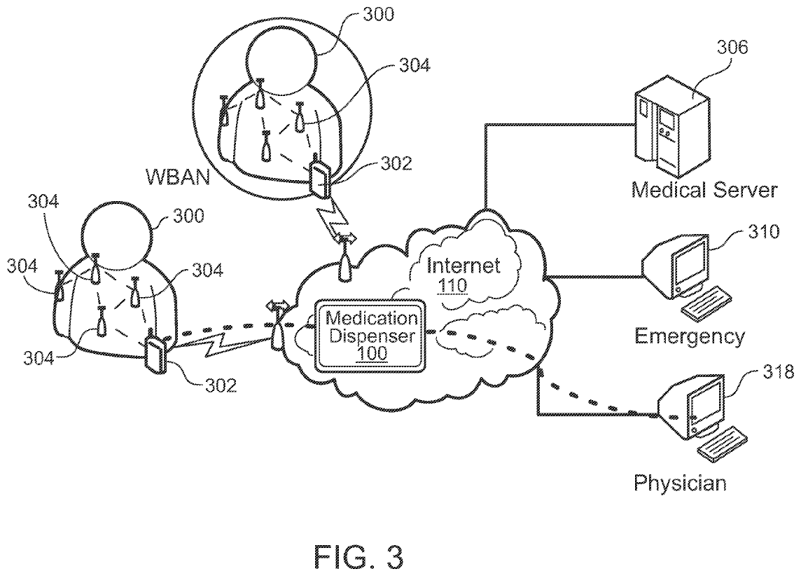

FIG. 4 illustrates a partial cut-away diagram of an example medication dispenser 100 in accordance with embodiments of the present disclosure. Referring to FIG. 4, the dispenser 100 includes a medication dispensing mechanism (not shown) for receiving pills at one of the receptacles 104. The dispenser 100 may include sensors (not shown) for identifying pills and for sorting the pills into one of the storage units 118 based on identification of the pills. For example, like pills can be sorted into the same storage unit 118. Thus, the storage units 118 may be dedicated to storing specific pills in a patient's regimen. When the medication manager determines to dispense a pill, the mechanism may cause a prescribed pill to be released from its storage unit 118 and into the holder 120.

During operation of the dispenser 100 shown in FIG. 4, a specific pill designated by a patient's regimen may be identified. A barcode and/or RFID on a pharmacy vial may be used to identify a specific pill, and the pill may be referenced to a pharmaceutical database that identifies its expected size, shape, weight, color(s), markings, generic name, and/or brand name. Verification of pills may be accomplished by any suitable equipment, such as computer vision. Based on identification of a specific pill to be handled, a stepper motor may rotate a carousel 400 carrying the storage units 118 such that a specific, designated storage unit 118 is positioned to receive pills for storage from the receptacle 104. It is noted that although only two storage units 118 are shown in FIG. 4, it should be understood that there may be any suitable number of storage units.

The dispenser 100 shown in FIG. 4 may utilize any suitable technique for sorting pills, such as a gravity based technique, motion, and a slotted opening. These techniques may be used to pass individual pills into a dedicated storage unit. In an example, components may be angled to thereby enhance the effects of gravity. A component may be rotated using a servomotor, thereby providing motion. Different speeds of rotation may be used to control the process. Small posts or bars 402 may be used to promote ricocheting to enhance dispersion of pills across a top surface 404. As a result of gravity and motion, pills may pass into receptacles 104 and drop onto a chute 406, which guides the pills into specific storage units 118. A suitable sensor using motion, light, weight, color, and/or the like may detect each pill as it passes down the chute 406 for determining the number of pills held by the storage unit. This information can be communicated to the medication manager for registration of information in a database. Upon a determination that all pills have been delivered from the receptacles 104, the servomotors may be instructed by the medication manager to discontinue rotation of the top surface 404. A mechanism may be used that ensures passage of only one type of pill into a designated storage unit 118. Segregation of pills may be accomplished based on the configuration of the direction and speed of rotation, angle of platform, size and shape of holes, placement of ricochet bars, or other functionalities. In an example, sensors may detect an unexpected pill delivery to a storage unit 118, and subsequently notify the medication manager for alerting an operator.

After all pills have been placed in their appropriate storage unit 118, pills may be dispensed for delivery to a patient at the holder 120. Particularly, after determining that a particular pill should be released according to a prescription schedule, the storage unit 118 storing the pill can be rotated by a servomotor such that the pill can drop through an opening 408 and down a chute 410. More than one storage unit 118 can be rotated in the case of two or more different pills being scheduled at the same time. Upon passage through the opening 408, the pill follows the chute 410 to the holder 120. Sensors can detect the passage of pills into or on the chute 410 and provide a signal of the detection to the medication manager, which may turn off the servomotor turning the carousel 400 when the proper number of pills has been delivered according to the prescription.

In example use, the dispenser may be stationary and adapted for use at home. FIGS. 2A-2D depict such an example dispenser 100. Referring to FIGS. 2A-2D, pills can be poured from their original bottles into a funnel on top of the dispenser 100 after registered by an embedded RFID reader 206. The pills may be counted and then be guided to one of the multiple storage units within the dispenser 100. At the time of administration of one or more pills, a dosage of pills according to the types and numbers in the schedule may be dispensed on-site into the holder 120. The dispenser 100 can detect whether the holder 120 has been open and whether the dosage of pills has been taken, creating a record of the medication event. If a user misses a dosage, no medicine is dispensed subsequently for that dosage. This method eliminates the wasting situation common in pre-sorted systems where a missed dosage needs to be discarded so that the next dosage can be dispensed. Compared to storing the medicine in their original bottles, this method presents better management in that the types and numbers of medicine to be taken are controlled, and a user has no way to abuse specific types of medicine or take medicine of incorrect types or number. This feature also enables the system to responsively accommodate dynamic medication scheduling or dispense as needed. For example, if the air quality deteriorates due to temporary events like pollens, a new medicine schedule may be generated for those taking medicine for respiratory disease, and the system can complete the updated dispensing task given the medicine is available.

The dispenser 100 includes a status lamp and pill delivery indicator 208 for indicating the availability of a pill in the holder 120. Further, the dispenser 100 may include a barcode reader 210 for reading mediation container barcodes to retrieve medication information about pills or other medication. The dispenser 100 may also include speakers 212 for communicating information in audio form to a patient or another. The dispenser 100 may also include an integrated camera 214 for capturing images and/or video of an operator of the dispenser 100.

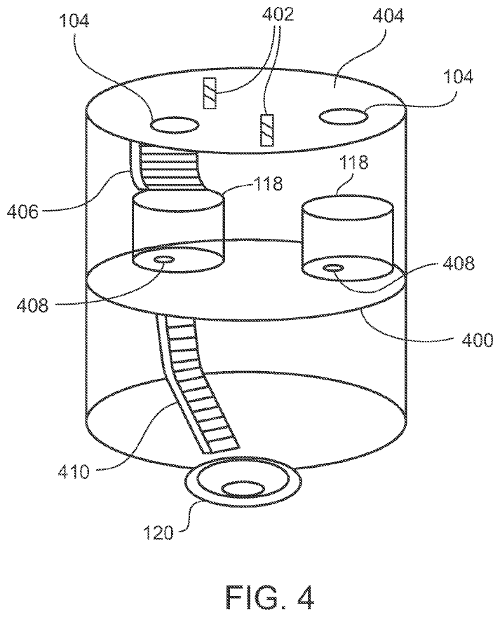

FIG. 5 illustrates a flow diagram depicting an example method of use of an example medicine administration system in accordance with embodiments of the present disclosure. The example method is depicted as being implemented by the dispenser 100 shown in FIGS. 2A-2D, although it should be understood that the method may be implemented by any suitable dispenser or system.

Referring to FIG. 5, initially at step 1, the dispenser 100 is turned on and made available for use by a patient. Subsequently, at step 2, an identification card 500 with an RFID tag may be waved in front of the RFID reader 206 for the dispenser to identify an authorized patient or other individual for use of the dispenser 100. The medication manager may receive the identifier from the RFID reader 206 and determine whether the individual is authorized. Other forms of user identification may also be employed, such as biometric identification, facial recognition, voice recognition, password input, and other methods. The user's use is declined if the individual is not authorized. In this example, it is assumed that the identification card 500 identifies an authorized individual, and therefore the medication manager permits use. In this case at step 3, the cover 200 may be opened manually or automatically by a suitable mechanism to allow the patient or other individual to put pills in the receptacle 104. The receptacle 104 may define an opening that leads to a container for holding the pills until the pills are sorted.

Subsequently at step 4, a pill bottle 502 containing pills to be placed in the receptacle 104 may be waved in front of the RFID or barcode reader 210 such that an RFID signal or barcode 504 on the bottle 502 can be read. The barcode 504 may include information identifying the pills and a prescription for the patient to take the pills. At step 5, the pills 506 may be poured into the receptacle 104. Step 6 depicts an interior of the dispenser 100 wherein a medication dispensing mechanism 508 is controlled by the medication manager to sort the pills 506 and place the identified pills into their specified storage unit 118. Step 7 depicts a last step in which the cover 200 is closed. In one or more steps of the method, the dispenser 100 may be in communication with a database 510 to send and retrieve data as described herein.

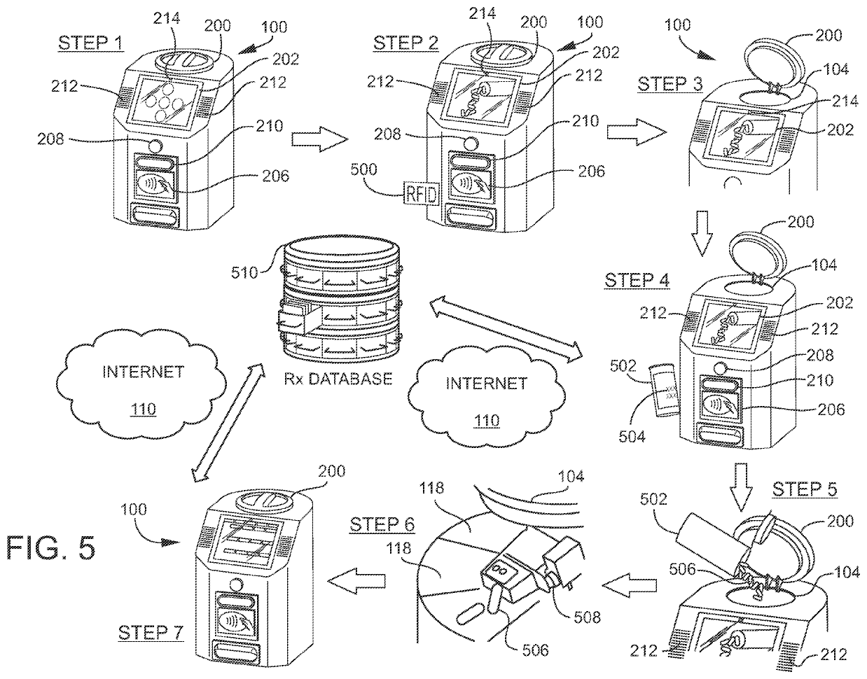

FIG. 6 illustrates a flow diagram depicting an example method of use of an example medicine administration system in accordance with embodiments of the present disclosure. The example method is depicted as being implemented by the dispenser 100 shown in FIGS. 2A-2D, although it should be understood that the method may be implemented by any suitable dispenser or system. In this example, the method is used for dispensing pills to a patient 600.

Referring to FIG. 6, initially at step 1, the dispenser 100 is turned on and made available for use by a patient. At step 2, the speakers 212 and/or the indicator 208 may activate to notify the patient that pills are available for consumption at the holder 120. At step 3, the pills 506 may be deposited into the holder 120, and the holder may be removed for access by the patient 600 (step 4). At step 5, the patient 600 may take the pills 506. At step 6, the holder 120 may be placed back inside the dispenser 100. At step 7, the dispenser 100 may communicate with the database 510 via the Internet to provide that the prescribed pill dosage has been taken by the patient 506.

A patient may stop the administration of a medication by overriding the automated control system, but under certain authorized circumstances (e.g., PRN medications), he or she is also be able to dispense a specific medication as needed.

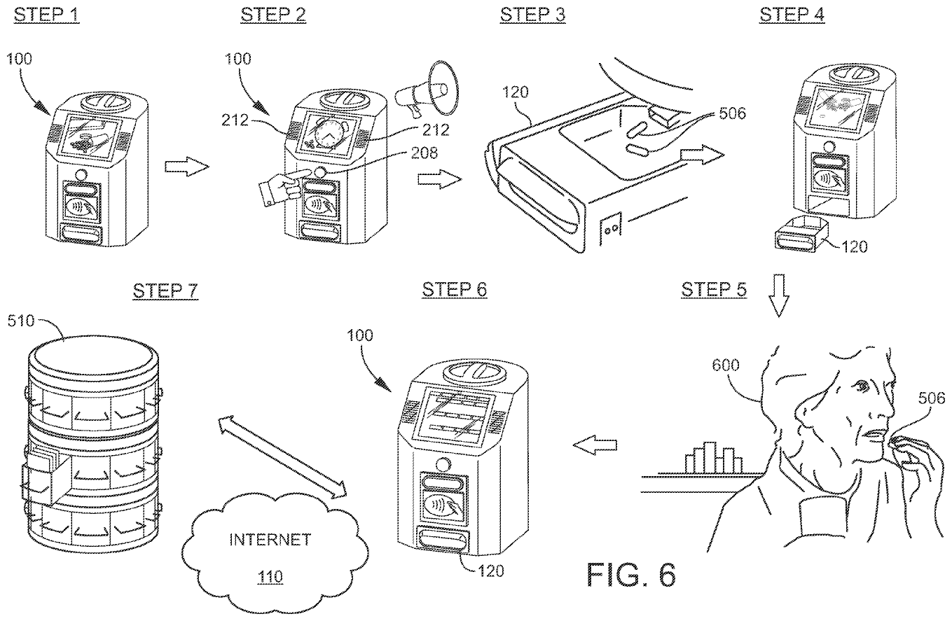

FIG. 7 illustrates a flow diagram depicting another example method of use of an example medicine administration system in accordance with embodiments of the present disclosure. The example method is depicted as being implemented by the dispenser 100 shown in FIGS. 2A-2D, although it should be understood that the method may be implemented by any suitable dispenser or system. In this example, the method is used for managing instances in which the patient 600 is not adhering to a prescription.

Referring to FIG. 7, initially at step 1, the dispenser 100 is turned on and made available for use by the patient 600. At step 2, the speakers 212 and/or the indicator 208 may activate to notify the patient that pills are available for consumption at the holder 120. At step 3, it may be determined that it is past the time when the patient 600 should have taken the pills. In response to determining that it is past the due time, the dispenser 100 may communicate via the Internet 110 to inform the database 510 or other computing device that the patient 600 is not adhering to a prescription (step 4). Subsequently, at step 5, a database server 510 may then communicate an email message to a computing device 700 of an assistant 702. Also, at step 5, the database server 510 may send an alert to a computing device 704 of healthcare personnel 706 (e.g., a physician), who may in turn send notification to a computing device 708 of the healthcare personnel's assistant 710. Subsequently, the healthcare personnel 706 or assistant 710 may record this information in an electronic record that may be tracked at a computing device 712 at step 6. At step 7, the electronic record may be routed to the dispenser 100 via the Internet 110.

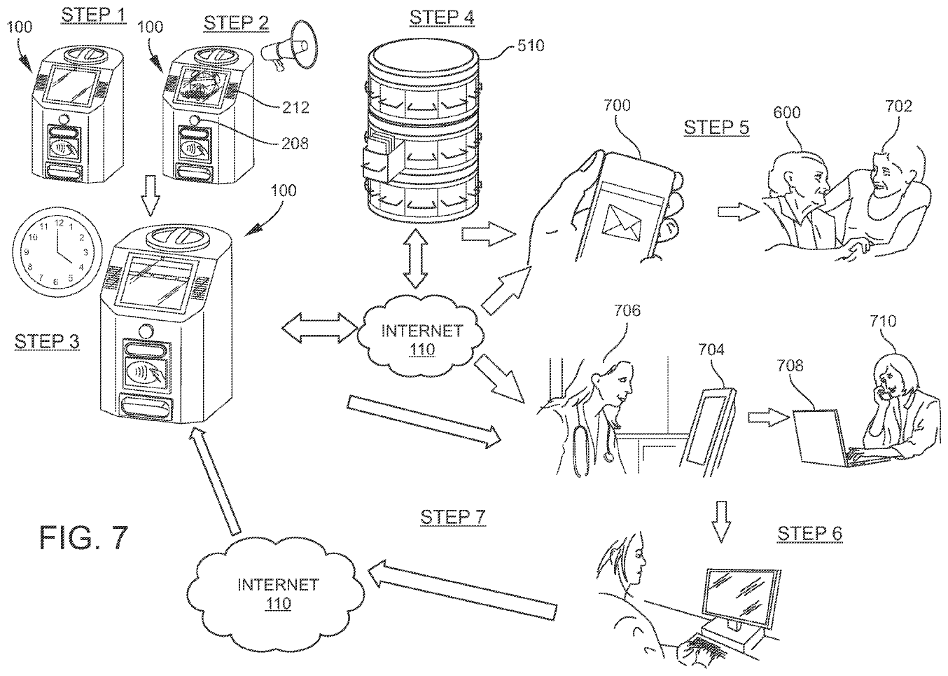

FIG. 8 illustrates a flow diagram depicting another example method of use of an example medicine administration system in accordance with embodiments of the present disclosure. The example method is depicted as being implemented by the dispenser 100 shown in FIGS. 2A-2D, although it should be understood that the method may be implemented by any suitable dispenser or system. In this example, the method is used for managing instances in which the pill count in the dispenser 100 is determined to be low.

Referring to FIG. 8, initially at step 1, the dispenser 100 may determine that the pill count is low. For example, the dispenser 100 may determine that the number of pills of a particular type is below a predetermined number (e.g., 5), or that the number of pills will only last three (3) more days based on the stored prescription. In response to this determination, the dispenser 100 may communicate a message to another computing device 800 (e.g., a smartphone) via the Internet 110. At step 2, the operator of the computing device 800 may read the message. At step 3, the dispenser 100 may also communicate a message to a computing device 802 of a physician 804 who authorizes a re-fill of the prescription associated with the pills. The operator of the computing device 800 may subsequently obtain a re-fill of the prescription at a pharmacy (step 4) and then proceed to re-fill the dispenser 100 with the pills for replenishing the supply (step 5).

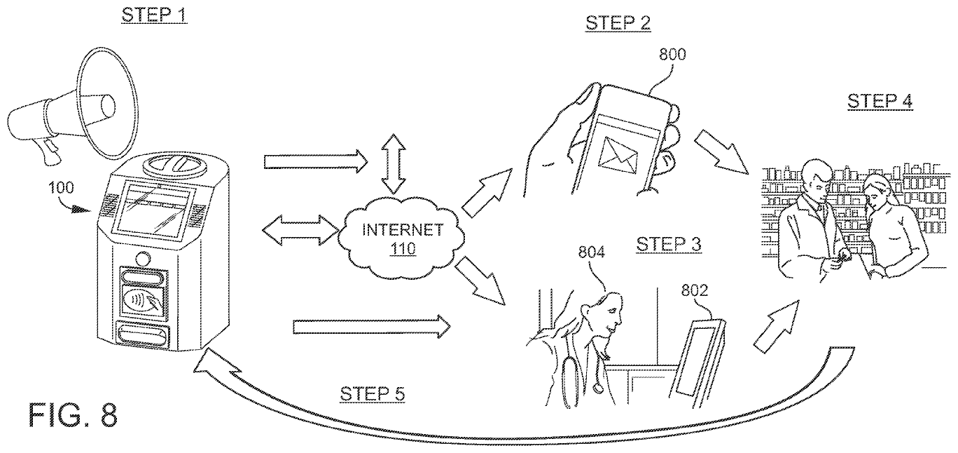

In accordance with embodiments, a dispenser may be in the form of a portable pillbox. As an example, the dispenser may be a case of smartphone that can supplement a medicine administration system as described herein. In an example, the case may include a number of compartments that can hold pills or other medication that the patient is scheduled to take during a predetermined period (e.g., a 24 hour period). FIG. 9 illustrates a diagram depicting an example case 900 and drawer 902 that cooperates with a dispenser 100 and mobile computing device 904 in accordance with embodiments of the present disclosure. Referring to FIG. 9, the drawer 902 defines three compartments 906A, 906B, and 906C. At step 1, a patient or other person may place the drawer 902 in a slot of the dispenser 100. The dispenser 100 may recognize insertion of the drawer 902 and subsequently control its display 202 to display information about pills 905A, 905B, and 905C that are to be placed in the compartments 906A, 906B, and 906C, respectively, of the drawer 902. These pills are the medication to be taken by the patient over a period of time in accordance with the patient's prescription. In this example at step 2, the display 202 indicates the identity and quantity of pills to be placed in the drawer 902. At step 3, the drawer 902 may be removed by the patient. Subsequently at step 4, the drawer 3 may be inserted in the case 904 attached to the computing device 904, a smartphone in this example. The dispenser 100 may synchronize information with the computing device 904 to notify the computing device 904 that the drawer 902 contains the pills. A display 908 of the computing device 904 may indicate contents of the drawer 902.

It is noted that the drawer 902 in this example contains no electronic components or battery itself, thus eliminating the problem of battery life and reducing its weight and cost. The computing device 904 may use an embedded camera (not shown) to monitor the status of each compartment 906A, 906B, and 906C via flat mirrors (not shown) placed at suitable locations. An application residing on the computing device 904 can process the captured image(s). Based on the monitoring of compartment status, the drawer 902 can enable the computing device 904 to remind a user to refill the compartments 906A, 906B, and 906C before leaving the location of the dispenser 100, to initiate an intake reminder, and to record a medication event automatically. The drawer 902 can also be integrated with the dispenser 100 in that the latter can dispense the dosages to be refilled manually into the compartments, or automatically complete the refill if the drawer 902 is located under the dispenser 100.

In accordance with embodiments, the computing device 908 can activate its embedded camera to capture one or more images of the three compartments 906A, 906B, and 906C. The captured image(s) may be processed to determine whether each compartment 906A, 906B, and 906C is empty. If permitted by the user, relevant data from other software applications can also be accessed, such as alarm, weather, calendar, and the like. Based on the information above, the application can provide a context-aware reminder for a user to refill the compartments 906A, 906B, and 906C before leaving home and to take the pills on time. These medication events can be recorded automatically by the computing device 904. The camera can provide automated detection of pill removal (e.g., by determining that a compartment previously filled is now empty) and transmit the information via the Internet 110 to the medication manager 106. The medication data together with the physiological data collected by a wearable device, for example, can be processed through a service management server, and corresponding services like family and medical support can be activated. The computing device 904 can be used as a reminder and an information exchange platform between user and a physician via a dedicated software application.

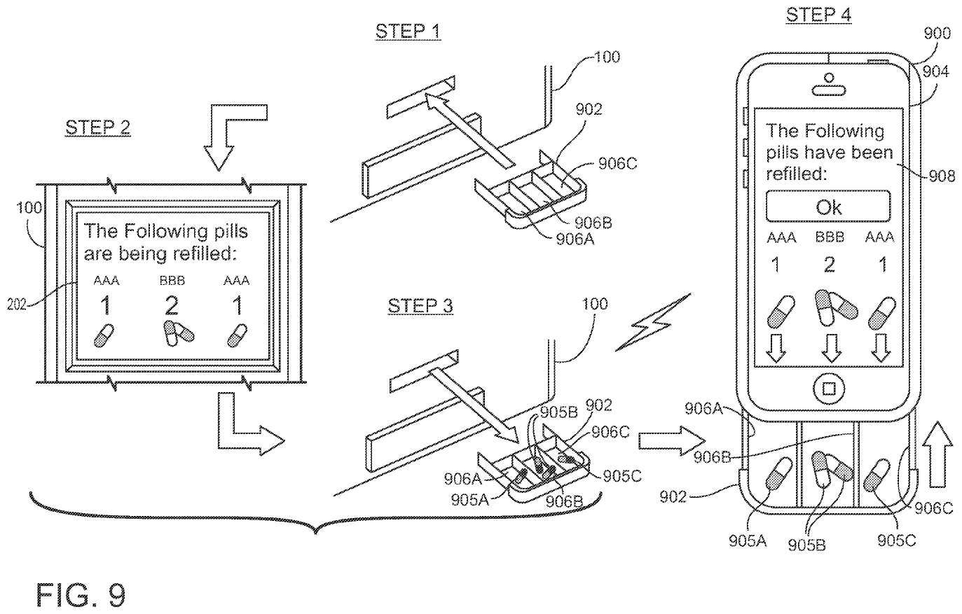

FIGS. 10A and 10B illustrate different views of the case 900 in accordance with embodiments of the present disclosure. Particularly, FIG. 10A illustrates an exploded view of the case 900 and shows the following components: a back component 1000. a front component 1002, and the drawer 902.

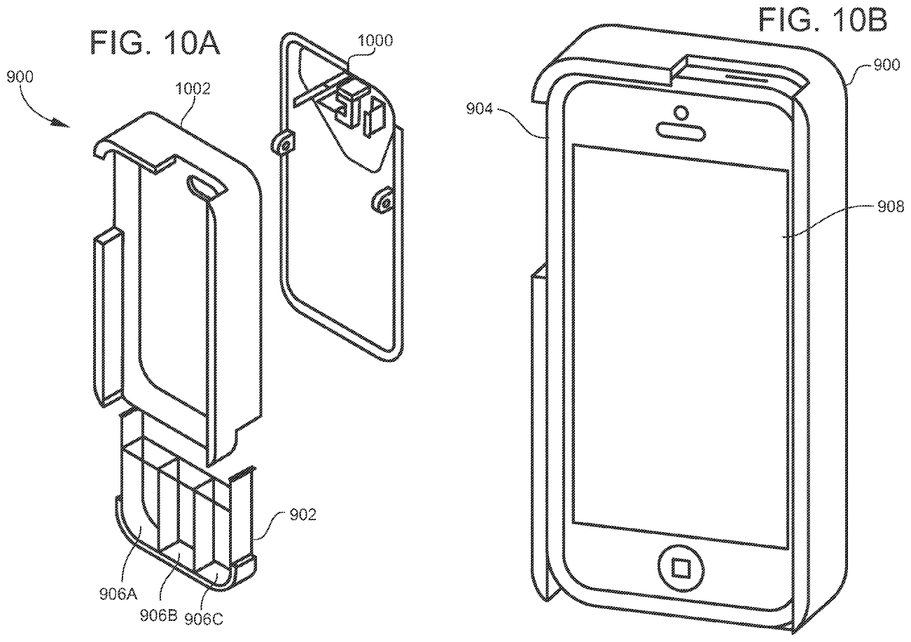

FIG. 11 illustrates a top view of an interior of the case 900 in accordance with embodiments of the present disclosure. Referring to FIG. 11, the case 900 can hold multiple mirrors 1100, 1102, and 1104 positioned such that the camera of a computing device covered by the case 900 can view the compartments 906A, 906B, and 906C. Mirror 1100 can be a flat mirror configured and positioned to change the view direction of the camera from +Z to +Y direction, while the second mirror 1102 enables the view to follow +X direction and cover all the three compartments 906A, 906B, and 906C. In an example, the size of a compartment can be up to 40 mm*18 mm*10 mm, which is sufficient to hold a normal dosage of medicine. The third mirror 1104 may be a flat mirror configured and positioned to guide the light along the +Y direction. The redirected light shares part of the second mirror 1102 and illuminates the three compartments 906A, 906B, and 906C. The first mirror 1100 may be positioned above the third mirror 1104 in the +Z direction, so that the view of the camera is not totally blocked by the third mirror 1104 and an image of illuminated pills can be captured.

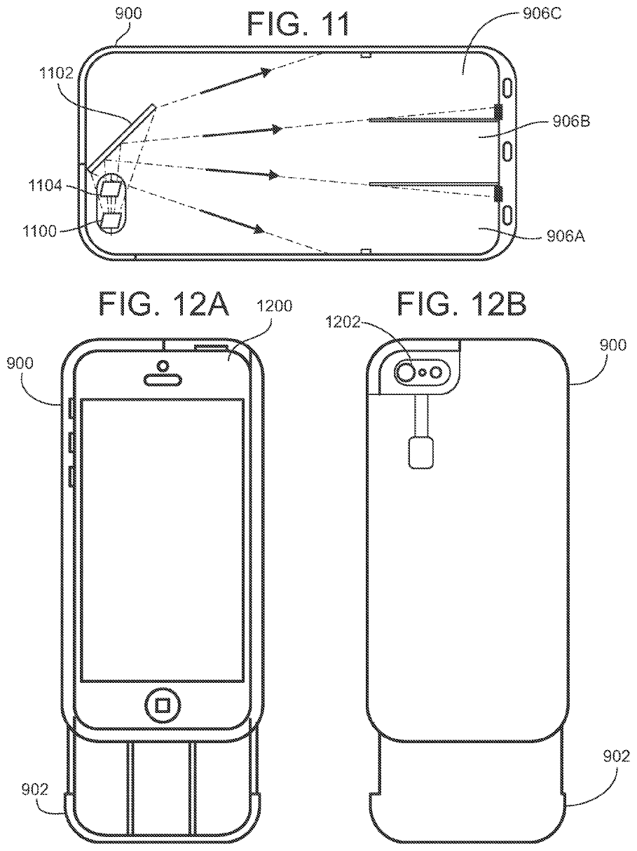

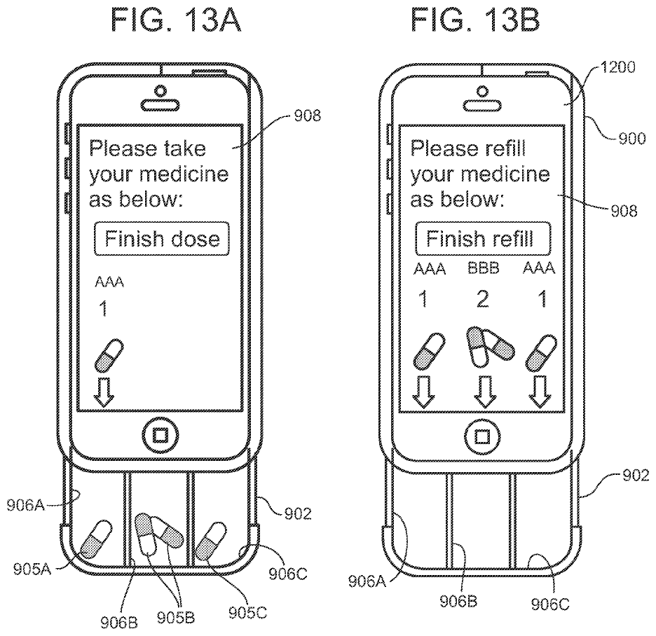

FIGS. 12A and 12B illustrate a front view and a rear view of a smartphone 1200 having a case attached thereto in accordance with embodiments of the present disclosure. In this example, the drawer 902 is in an open position. The three mirrors are placed on the support structures over a sliding pad as shown. Opaque blockers can be placed around the third mirror 1104 to better control the scattering of the LED light and the field of view in the first mirror 1100. By moving the pad along a groove on the case 900 as shown in FIGS. 12A and 12B, the camera and the LED light can still be uncovered and used to take photos as usual through the opening. The drawer's upper wall can be made of transparent plastic material, so a camera 1202 can still see through it to monitor the status of each compartment, and the LED light can illuminate. This design of drawer 902 is useful for users to associate the information provided in the phone screen with the manipulations related to individual compartments. When the smartphone software is guiding a user to reload pills, it can clearly show the types and quantities of the pills 905A, 905B, and 905C to be put into a specific compartment, as is shown in FIGS. 13A and 13B, which illustrates top views of the case 900 and smartphone 1200. At the time of reminding a user to take the pills 905A, 905B, and 905C, a displayed arrow 1300 on the display 908 can provide intuitive and accurate guidance with the number and quantities of the pills to be taken as shown in FIGS. 13A and 13B. If a user also has a stationary medication system, the pill refill process can become highly automatic as described. The stationary system may subsequently display related information on its display and dispense the required pills into the three compartments 906A, 906B, and 906C of the drawer as three doses according to schedule. Once this process is finished, the drawer 902 can be detached and put back into the case 900. Meanwhile, the stationary system can sychronize the pill information with the software application on the smartphone wirelessly.

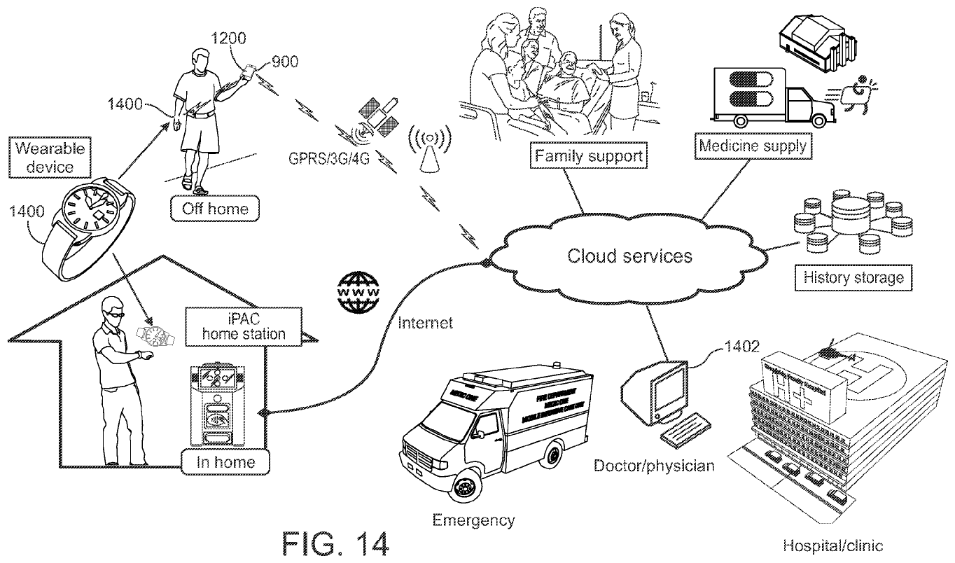

In accordance with embodiments, a system may operate with a wearable device that monitors relevant physiological data of a user, e.g. heart rate, body temperature, and the like. For example, FIG. 14 illustrates a flow diagram of a patient using a wearable device 1400 in accordance with embodiments of the present disclosure. The wearable device 1400 is a watch in this example and can be connected via Bluetooth with the stationary system or a smartphone 1200 having a case 900 as described herein. The smartphone can be used to transfer the data to a physician's computer 1402. In addition to providing the monitoring of the health status, these data can serve as feedback for evaluation of the medication and determination of necessary reactions. The updated medication schedules can in turn be implemented with the stationary system and the portable pillbox.

With this integrated system, (1) medication management can be more effective and efficient, (2) the medication schedules can be followed in a more timely and accurate manner, (3) medication events can be recorded more reliably, and (4) fast medical responses can be achieved via post-medication feedback. As a result, an environment with more comprehensive medication management and healthcare can be established. Medication data can be collected by the home station and the cellphone case for in-home and away-from-home situations, respectively. These data include the time, type and number of each prescribed dosage, as well as information about inventory and refill. Together with the physiological data collected from the wearable device via Bluetooth, the medication data are transmitted through the Internet or cellular networks to the Cloud, which provides a variety of services. The family members of a patient can learn about his/her health conditions and medication adherence level via email or other notifications, so that they can give timely interventions, encouragement, and support which are no less important than a medication treatment itself. Remote databases store the longitudinal data of each specific patient. A physician can inspect the medication history and physiological status history of a specific patient to evaluate the recovery progress and prepare a new prescription accordingly. By using dedicated software, a physician can also effectively manage a large group of patients. Emergency centers and hospitals/clinics can better allocate their resources to serve the patients who need help most. Medicine providers can learn about the consumption of their products by patients, so that they can provide corresponding services such as door-to-door delivery of refill medicine. if some type of medicine turns out to lead to serious side effects across a large group of users, pharmaceutical companies can become aware earlier and a warning or recall can be carried out in a timelier manner.

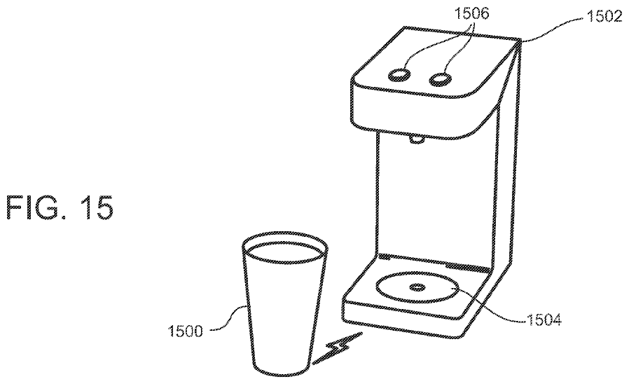

In accordance with embodiments, FIG. 15 illustrates a diagram of a system having a sensor-embedded water cup 1500 for medication adherence detection according to embodiments of the present disclosure. Referring to FIG. 15, to detect a patient taking pills, an additional feature of sensor-embedded water cup 1500 design can be used. The cup 1500 can be integrated with systems for medicine-taking detection. A water cup station 1502 can have a weight sensor 1504 which can be used to determine whether a cup is thereon and/or how much water is taken. The station may be connected to the sensor of the cup 1500 via Bluetooth or any other suitable wireless connection. Further, the station 1502 may be connected to another computing device or a network via any suitable wireless connection. The station 1502 may include water delivery buttons 1506. Further, the station 1502 may be integrated with a water filter, water magnetization device, and/or water flow sensors. The water flow sensors can be used for monitoring a user's daily water intake.

In an example use of the station 1502, medicine may be dispensed in a drawer as described. Subsequently, a cup 1500 may be taken from the station 1502. A drinking action may be detected based on the cup sensor. Detection of the drinking action may be transferred to the station and subsequently sent to a system for recording.

In accordance with embodiments, a computing device (e.g., dispenser) or system as disclosed herein can detect non-adherence of a patient to a prescription. Based on this, the non-adherence may be addressed. For example, if it were detected that the patient is non-adherent to large pills but adherent to small pills, the computing device or system can detect this pattern after several medication administrations and then address it, for example, before dispensing a large pill providing a message on the interface noting that this particular pill he/she does not like to take and—assuming a gaming mode—perhaps providing an incentive within the game to ingest it (extra points). Alternatively, the computing device or system can remind the patient that this drug is critical to his or her medical condition or notify his or her physician that the pill is too large and that another, smaller pill exists for the same indication. In this way, for example, data collected by the computing device or system can be used in an artificially intelligent manner to tailor processes to the individual patient.

In accordance with embodiments, a computing device (e.g., dispenser) or system as described herein may be used in conjunction with other medical equipment. For example, taking hypertension as an indication for example, if the medication is prescribed at the wrong dosage and the patient becomes hypotensive, this information can be detected by an attached blood-pressure monitor, and the system may subsequently reduce the dosage. This may be reported to a physician such that the physician can be involved in the decision making for complex issues. Over time the data may be used for machine learning and big-data analyses for the continuous accumulation in real time of large amounts of data on drugs and their efficacy using instrumentation, such as blood-pressure readings, to create medical knowledge and make expert decisions.

It is also noted that a computing device (e.g., dispenser) as described herein may be AC powered, but a DC backup may also be utilized.

The various techniques described herein may be implemented with hardware or software or, where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programble computers, the computer will generally include a processor, a storage medium readable by the processor (including volatile and non-volatile memory and/or storage elements), at least one input device and at least one output device. One or more programs may be implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims. One skilled in the art will readily appreciate that the present subject matter is well adapted to carry out the objects and obtain the ends and advantages mentioned, as well as those inherent therein. The present examples along with the methods described herein are presently representative of various embodiments, are exemplary, and are not intended as limitations on the scope of the present subject matter. Changes therein and other uses will occur to those skilled in the art which are encompassed within the spirit of the present subject matter as defined by the scope of the claims.

1-73. (canceled)

74. A medicine administration system comprising:
a case having a drawer that defines a plurality of compartments for holding medication; and
a mobile computing device comprising:
a housing configured to attach to the case;
a user interface; and
at least one processor and memory configured to:
determine contents of each of the compartments;
manage adherence to a prescription based on the contents; and
control the user interface to present information to a user about adhering to the prescription and information about location of prescribed medication in the compartments.

75. The medicine administration system of claim 74, wherein the mobile computing device is a smartphone.

76. The medicine administration system of claim 74, wherein the mobile computing device comprises a camera configured to capture images of the contents of the compartments, and
wherein the at least one processor and memory are configured to determine the contents of the compartments based on the captured images.

77. The medicine administration system of claim 74, wherein the user interface comprises a display, and
wherein the at least one processor and memory are configured to control the display to display indicators about the contents of the compartments.

78. The medicine administration system of claim 74, wherein the at least one processor and memory are configured to control the display to display information about adhering to the prescription for the medication held by the compartments.

79. The medicine administration system of claim 74, wherein the at least one processor and memory of the mobile computing device are configured to:
receive physiological data of the user;
integrate the physiological data with adherence data to enhance medical care; and
control the user interface to present the information based on the physiological data and adherence data.

80. The medicine administration system of claim 79, wherein the at least one processor and memory of the mobile computing device are configured to receive the physiological data from a wearable device and integrate the physiological data with the adherence data to enhance medical care.

81. A medicine administration system comprising:
a case having a drawer that defines a plurality of compartments for holding medication;
a mobile computing device comprising:
a housing configured to attach to the case;
a user interface; and
at least one processor and memory configured to:
determine contents of each of the compartments;
manage adherence to a prescription based on the contents;
communicate, to another computing device, a notification that the medication in accordance with the prescription has been dispensed;
verify the identify of the patient intended to receive the medication; and
communicate, to the other computing device, a notification that an action has been taken by the verified patient.

82. The medicine administration system of claim 81, wherein the mobile computing device is a smartphone.

83. The medicine administration system of claim 81, wherein the mobile computing device comprises a camera configured to capture images of the contents of the compartments, and
wherein the at least one processor and memory are configured to determine the contents of the compartments based on the captured images.

84. The medicine administration system of claim 81, wherein the user interface comprises a display, and
wherein the at least one processor and memory are configured to control the display to display indicators about the contents of the compartments.

85. The medicine administration system of claim 81, wherein the at least one processor and memory are configured to control the display to display information about adhering to the prescription for the medication held by the compartments.

86. The medicine administration system of claim 81, wherein the drawer is configured to engage with a medication dispenser for receipt of medication within one or more of the compartments, and
wherein the at least one processor and memory are configured to control the display to display information about the medication placed within the one or more of the compartments.

87. The medicine administration system of claim 86, wherein the at least one processor and memory are configured to receive user input indicating acknowledgement of view of the displayed information about the medication.

88. The medicine administration system of claim 86, wherein the at least one processor and memory are configured to control the display to identify a medication contained in one or more of the compartments.

89. The medicine administration system of claim 86, wherein the at least one processor and memory are configured to receive user input indicating adherence to a prescription for medication contained in one or more of the compartments.

90. The medicine administration system of claim 86, wherein the at least one processor and memory are configured to control the display to indicate one or more of the compartments to be filled with medication by the user.

91. The medicine administration system of claim 86, wherein the at least one processor and memory are configured to display prescription adherence information to a user based on a view of contents of the compartments acquired from an image capture device.

92. The medicine administration system of claim 86, wherein the prescription adherence information includes information about refilling one or more compartments.

93. A mobile computing device case comprising:
a drawer that defines a plurality of compartments for holding medication; and
a housing attached to the drawer and being configured to hold to a mobile computing device.

94. The mobile computing device case of claim 93, wherein the drawer is integrated with the housing and configured to be positioned in a first position or a second position with respect to the housing,
wherein in the first position, interior spaces of the compartments are at least substantially closed such that medication located therein fall outside of their respective compartments, and
wherein in the second position, interior spaces of the compartments are accessible from outside such that medication located therein are accessible.

95. The mobile computing device case of claim 93, further comprising at least one mirror positioned to reflect images of contents of the compartments towards a camera of a mobile computing device attached to the housing.

96. The mobile computing device case of claim 95, wherein the drawer has a transparent portion such that the contents of the compartments are within view of the camera.

97. The mobile computing device case of claim 93, wherein the drawer is configured to engage with a medication dispenser for receipt of medication within one or more of the compartments.
